(19) United States
(12) Patent Application Publication
(10) Pub. No.: US 2018/0368445 A1
(43) Pub. Date: Dec. 27, 2018

(54) LIVE CYANOBACTERIA AS A BINDING AGENT IN HUMAN AND ANIMAL FOODS

(71) Applicant: Lawrence V. Dressler, Cranston, RI (US)

(72) Inventor: Lawrence V. Dressler, Cranston, RI (US)

(21) Appl. No.: 16/017,552

(22) Filed: Jun. 25, 2018

Related U.S. Application Data
(60) Provisional application No. 62/524,176, filed on Jun. 23, 2017.

(51) Int. Cl. A23K 10/16; A23L 29/00; C12N 1/20; A23L 33/135; A23L 3/005; A23L 5/10

(52) U.S. Cl. CPC A23K 10/16; A23L 29/065; A23L 5/10; A23L 33/135; A23L 3/005; C12N 1/20

(57) ABSTRACT

A method and system for using live cyanobacteria as a binding agent in human and animal foods. A method includes growing live cyanobacteria in closed bio-reactors, harvesting the grown living cyanobacteria in a filtration system to create a pudding-like consistency, mixing the harvested living cyanobacteria with dry and wet ingredients to create a batter, forming the batter into a desired shape, and dehydrating the shaped batter using low heat.

### CROSS REFERENCE TO RELATED APPLICATIONS

This application claims benefit from U.S. Provisional Patent Application Ser. No. 62/524,176, filed Jun. 23, 2017, which is incorporated by reference in its entirety.

### STATEMENT REGARDING GOVERNMENT INTEREST

None.

### BACKGROUND OF THE INVENTION

The invention generally relates to human nutrition and the pet food industry, and more specifically to live cyanobacteria as a binding agent in human and animal foods.

A common trend in human nutrition and pet food industry is “limited ingredients.” Consumers are looking for transparency in terms of wholesomeness of ingredients, lack of artificial ingredients, lack of common allergens, lack of fillers and binders. Issues arise when trying to keep formed food/feed together without adding in a variety of low nutrition ingredients that have binding features. For example potato starch and flour are common binders but they contain minimal nutritional value, protein and fat. Most formed foods or feed are cooked and then pressed into form. When it comes to animal feed a common finished product are pellets. Pellets are formed when ingredients, starch binders and moisture are cooked at high temperatures and then extruded under high pressure. The purpose of cooking at high temperatures is to breakdown the starch so that it can create a “plastic” like structure when it cools. This structure allows for the finished product to keep its form during packaging, warehousing and transportation.

### SUMMARY OF THE INVENTION

The following presents a simplified summary of the innovation in order to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later.

In general, in one aspect, the invention features a method including growing live cyanobacteria in closed bio-reactors, harvesting the grown living cyanobacteria in a filtration system to create a pudding-like consistency, mixing the harvested living cyanobacteria with dry and wet ingredients to create a batter, forming the batter into a desired shape, and dehydrating the shaped batter using low heat.

Embodiments may include drying the dehydrated batter for a pre-set time and cooling the dried dehydrated batter.

In another aspect, the invention features a system including a closed bio-reactor configured to grow live cyanobacteria, a filtration device configured to harvest the grown living cyanobacteria to create a pudding-like consistency, a mixing device configured to mix the pudding-like living cyanobacteria with dry and wet ingredients to create a batter, a shaping device configured to form the batter into a desired shape, and a dehydration device configured to dehydrate the shaped batter using low heat.

Embodiments may include a drying device, the drying device configured to dry the dehydrated batter for a pre-set time, and a cooling device, the cooling device configured to cool the dried dehydrated batter.

The invention may include one or more of the following advantages.

The present invention uses live cyanobacteria (e.g., spirulina), so that when formed batter is drying at low heat, cell walls start to breakdown releasing natural carbohydrates. These natural carbohydrates, made of simple sugars and proteins, leach out from the live cyanobacteria onto the other ingredients. When the product starts to cool the sugars have a sticky consistency and upon full cooling the cyanobacteria carbohydrates act as a binder.

The present invention uses no dry spirulina/cyanobacteria powder.

These and other features and advantages will be apparent from a reading of the following detailed description and a review of the associated drawings. It is to be understood that both the foregoing general description and the following detailed description are explanatory only and are not restrictive of aspects as claimed.

### BRIEF DESCRIPTION OF THE DRAWINGS

These and other features, aspects, and advantages of the present invention will become better understood with reference to the following description, appended claims, and accompanying drawings where:

FIG. 1 is a flow diagram.

FIG. 2 is a block diagram.

### DETAILED DESCRIPTION

The subject innovation is now described with reference to the drawings, wherein like reference numerals are used to refer to like elements throughout. In the following description, for purposes of explanation, numerous specific details are set forth in order to provide a thorough understanding of the present invention. It may be evident, however, that the present invention may be practiced without these specific details. In other instances, well-known structures and devices are shown in block diagram form in order to facilitate describing the present invention.

The present invention addresses how to make limited ingredient, nutritious human food or animal feed without adding low nutrition binders, and using high energy (e.g., steam), high heat, high pressure.

Spirulina is commonly referred to as an edible blue-green algae but more correctly it is a cyanobacteria. It is also considered an aquatic micro-organism because it is grown in mostly brackish water. Spirulina is considered a “Superfood” because it is high in protein (e.g., 55-70%), nutrient dense, highly digestible with anti-inflammatory benefits. Both NASA and World Health Organization consider Spirulina to be among the healthiest known organisms. Spirulina is a whole food and complete protein. Whole food refers to a plant food that is unprocessed and unrefined or that which has had minimum processing and/or refining. Complete protein is a source of protein that contains all nine of the essential amino acids as well as non-essential amino acids. This is not common among plants but Spirulina meets this definition.

Spirulina consists of approximately 55-70% protein, 15-28% carbohydrates, 0.5-8% lipids and moisture. Spirulina has no root system and is grown in water, not hydroponically or in soil. It has soft cell walls made of complex sugars and protein that can breakdown quickly with low heat or by human/animal digestive systems.

The present invention describes how to grow and harvest edible, nutritious living cyanobacteria (e.g., spirulina). When this living cyanobacteria is harvested the excess water is drained leaving a pudding-like consistency. Other wet and/or dry ingredients are mixed with the wet living cyanobacteria at ambient temperature and at low speeds to make a batter. The batter can hand formed or pressed flat at low pressure. The formed batter is then placed inside of a dehydrator, which is used for drying. A dehydrator refers to a device that removes moisture from food to aid in its preservation. The dehydrator is set at a low drying temperature typically between 110° F.-125° F. The industry standard for cyanobacteria drying is a spray-drying process, which uses higher temperature and pressure and can negatively affect amino acids and nutrients.

When the formed batter is drying at low heat, the cell walls start to breakdown releasing the natural carbohydrates. These carbohydrates, made of simple sugars and proteins, leach out from the live cyanobacteria onto the other ingredients. When the product starts to cool the sugars have a sticky consistency and upon full cooling the live cyanobacteria carbohydrates act as a binder.

The present invention enables the production of limited ingredients food/feed products when one combines fresh, harvested wet living cyanobacteria (high protein & nutrient dense) with other wet and dry ingredients, mixed and dried under low temperatures. There is no need for high energy, high temperature, high pressure, which can damage nutrients and amino acids.

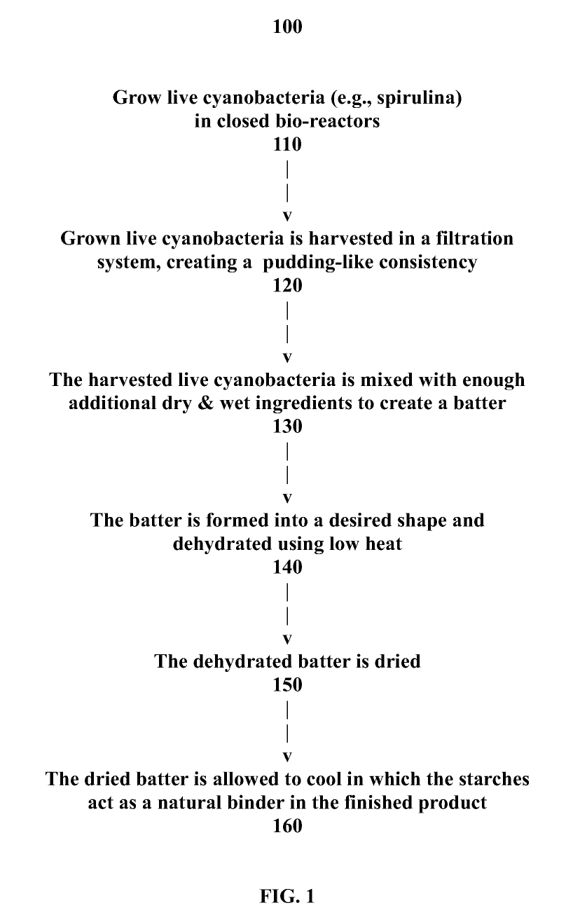

Turning now to FIG. 1, a method 100 of producing live cyanobacteria as a binding agent in human and animal foods includes growing (110) cyanobacteria (e.g., spirulina) in closed bio-reactors.

Grown living cyanobacteria is harvested (120) in a filtration system, creating a pudding-like consistency in which the cyanobacteria is still alive.

The harvested living cyanobacteria is mixed (130) with enough additional dry & wet ingredients to create a batter. The dry and wet ingredients may be feed ingredients or food ingredients. Example feed ingredients include, wet ingredients such as vegetable or fish oils and/or water, and dry ingredients such as grain meals, fish meal, shrimp meal, kelp (seaweed) and protein powders. Example food ingredients include wet ingredients such as edible vegetable oils and/or water, and dry ingredients such as nuts, seeds, dried fruit, cinnamon and seasonings.

The batter consisting of live cyanobacteria is formed (140) into a desired shape and dehydrated using low heat, such as heat between 110° F. and 125° F. The low heat causes natural carbohydrates (starches) to leach out of cells onto other ingredients. The batter may be formed (140) by hand or automatically, such as in a food processor.

The dehydrated batter is dried (150) for a specified amount of time. For example, depending on ingredients, drying time may be three to eight hours, allowed to cool (160) in which the starches act as a natural binder in the finished product.

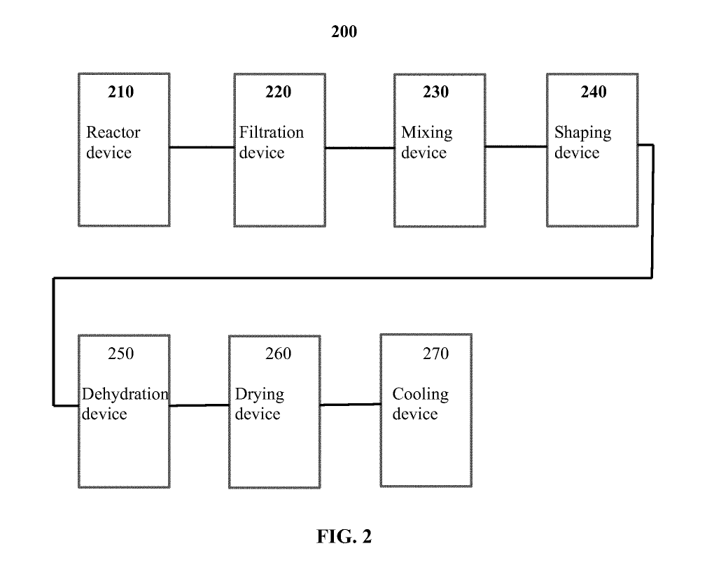

As shown in FIG. 2, an exemplary system 200 producing live cyanobacteria as a binding agent in human and animal foods include a closed bio-reactor 210 configured to grow live cyanobacteria and a filtration device 220 configured to harvest the grown living cyanobacteria to create a pudding-like consistency.

The system 200 includes a mixing device 230 configured to mix the pudding-like living cyanobacteria with dry and wet ingredients to create a batter and a shaping device 240 configured to form the batter into a desired shape.

The system 200 includes a dehydration device 250 configured to dehydrate the shaped batter using low heat.

The system 200 includes a drying device 260, the drying device 260 configured to dry the dehydrated batter for a pre-set time and a cooling device 270, the cooling device 270 configured to cool the dried dehydrated batter. In one embodiment, the drying device 206 and cooling device 27 are implemented as a single device configured to dry and cool.

It would be appreciated by those skilled in the art that various changes and modifications can be made to the illustrated embodiments without departing from the spirit of the present invention. All such modifications and changes are intended to be within the scope of the present invention except as limited by the scope of the appended claims.

What is claimed is:

1. A method comprises:
growing live cyanobacteria in closed bio-reactors;
harvesting the grown living cyanobacteria in a filtration system to create a pudding-like consistency;
mixing the harvested living cyanobacteria with dry and wet ingredients to create a batter;
forming the batter into a desired shape; and
dehydrating the shaped batter using low heat.

2. The method of claim 1 further comprising drying the dehydrated batter for a pre-set time, where cell walls start to breakdown releasing the natural carbohydrates, the natural carbohydrates, made of simple sugars and proteins, leaching out from the live cyanobacteria onto other ingredients.

3. The method of claim 2 further comprising cooling the dried dehydrated batter to produce a sticky consistency wherein the cyanobacteria carbohydrates act as a binder.

4. The method of claim 3 wherein the cooling is performed for a specified amount of time.

5. The method of claim 4 wherein the specified amount of time is 2-4 hours.

6. The method of claim 1 wherein the heat is between 110° F. and 125° F.

7. A system comprising:
a closed bio-reactor configured to grow live cyanobacteria;
a filtration device configured to harvest the grown living cyanobacteria to create a pudding-like consistency;
a mixing device configured to mix the pudding-like living cyanobacteria with dry and wet ingredients to create a batter;
a shaping device configured to form the batter into a desired shape; and
a dehydration device configured to dehydrate the shaped batter using low heat.

8. The system of claim 7 further comprising a drying device, the drying device configured to dry the dehydrated batter for a pre-set time.

9. The system of claim 8 further comprising a cooling device, the cooling device configured to cool the dried dehydrated batter.

10. The system of claim 9 wherein the cooling device is enabled for a specified amount of time.

11. The method of claim 10 wherein the specified amount of time is 2-4 hours.

12. The system of claim 7 wherein the heat is between 110° F. and 125° F.
